(19) United States
(12) Patent Application Publication
(10) Pub. No.: US 2006/0258008 A1
(43) Pub. Date: Nov. 16, 2006

(54) Method for testing the impermeability of components that contain cavities

(76) Inventors: Helmuth Holler, Kallham (AT); Walter Alfred Stephan, Martin (AT)

Correspondence Address: JACOBSON HOLMAN PLLC, 400 SEVENTH STREET N.W., SUITE 600, WASHINGTON, DC, 20004, US

(21) Appl. No.: 10/554,700

(22) PCT Filed: Apr. 29, 2004
(86) PCT No.: PCT/AT04/00143

§ 371 Date: Oct. 27, 2005

(30) Foreign Application Priority Data
Apr. 29, 2003 (AT) A 657/2003

(51) Int. Cl. G01N 33/00

(52) U.S. Cl. 436/3

(57) ABSTRACT

The invention relates to a method for testing the impermeability of components (1) that contain cavities (7). On at least one side of the component (1) to be tested, at least the surface to be tested is completely wetted with a foam-producing testing liquid (11). The aim of the invention is to create a method that can be carried out as quickly and easily as possible, during which the component (1) to be tested is influenced as little as possible by the testing liquid (11). To this end, the component (1) is subjected to an increase in temperature, and the surface of the component (1) to be tested is subsequently examined with regard to the formation of bubbles (12) in the testing liquid (11). The testing liquid (11) preferably consists of 10 to 20% dissolved surfactants, 2 to 6% alcohol, and 75 to 78% water and a maximum of 5% auxiliary agents.

The invention relates to a method for leak-testing components having cavities, wherein at least on one side of the component to be tested, at least the area to be tested is completely wetted with a foam-forming testing liquid.

Basically, the subject method and the testing liquid may be applied to a wide variety of components which have cavities. The method described and the testing liquid described particularly are used when leak-testing components made of synthetic composite materials, particularly with lightweight cores. Such components made of synthetic composite materials with light-weight cores in the form of honeycomb parts are particularly employed in aircraft technology. Such components have a particularly low weight and simultaneously particularly good strength properties.

For a quality control of components for aircraft, the latter often are scanned by means of ultrasonics. Such methods are highly complex and time-consuming. Moreover, by means of ultrasonics the leak-tightness of the components cannot be checked.

To leak-test components, in particular components of aircraft, e.g. gas is introduced into the interior of the fuselage, and the outer shell is scanned with a gas sensor. Such a method is described in U.S. Pat. No. 4,976,136 A, e.g. Yet, this method is particularly complex and is mainly suitable for closed spaces into which the gas can be introduced.

A further method for measuring a leakage, in particular in tanks, is described in WO 02/01175 A1, wherein the tank is circumferentially sealed and the air between the cover and the surface of the tank is removed by suction. In case of a leak of the tank, it will not be possible to maintain this vacuum between the circumferential seal and the tank surface. Apart from the great expenditures of this method, a precise localization of the leak is not possible with this method.

DD 140 172 A describes a method for locating leaks by means of foaming agents, in which pressurized devices, in particular telecommunication cables, are provided with a soap solution and possible leaks can be detected by means of the foam accumulation forming. For this method it is necessary for the interior of the body to be tested, the inner space of a telecommunication cable in the present case, to be pressurized relative to the environment. When leak-testing, with some components, such as, e.g. components made of synthetic composite materials with lightweight cores, this is not possible.

CA 2 148 844 A shows a method and a device for leak-testing air barriers in buildings, wherein a type of suction bell is put over the area of the air barrier to be tested, and a negative pressure is produced by means of a fan. The area to be tested is covered by brushing on a foaming material, whereby leaks can be optically detected due to the formation of bubbles.

In the aircraft industry, also immersion methods are used for leak-testing components made of a synthetic composite material, wherein the synthetic composite material component is immersed in a liquid-filled basin, and the liquid is heated. Due to the temperature increase, the expanded air present in the cavities of the components builds up a pressure, whereupon the air will escape at leaks possibly present. The escape of air is recognized by bubbles rising in the liquid container. The leaks are localized, and will be marked after the component has been lifted out of the liquid container. Apart from the complex handling, in particular with large components, this method does not allow for a rapid and reliable identification of the flaws. Moreover, heating of the water bath is particularly energy and time consuming. Furthermore, there exist regulations according to which the components may remain only shortly within water basins, e.g. 30 s at the most. With large components, this time mostly is not sufficient for locating flaws, and therefore the procedure must be repeated as often as necessary. Immediately after the component has been removed from the bath, the former must be rubbed dry so as to prevent moisture from being sucked in during cooling of the structure.

After the detection of leakages in such components, in particular in sandwich-type synthetic material bodies, the latter are repaired, if possible, and then the leakage test is repeated. The repairs and the renewed tests are repeated until no further leaks are found on the component. Subsequently, the component tested is marked accordingly and released for further use.

The present invention has as its object to provide a method for leak-testing components having cavities, in particular components made of synthetic composite materials and having honeycomb cores, which can be carried out as rapidly and easily as possible, and in which the component to be tested is influenced as little as possible. Furthermore, with the method according to the invention it shall also be possible to leak-test merely parts of the components, without having to test the entire component in each such test. Moreover, the method shall allow for a rapid, reliable and precise identification of the flaws.

The object according to the invention is achieved in that the component is subjected to a temperature increase and in that subsequently the component's area to be tested is checked for a bubble formation of the testing liquid. In contrast to known methods, in the subject testing method a rapid and simple leakage check is possible. Since the component to be tested is not submersed in water, also a more rapid, more reliable and more precise identification of the flaws and, consequently a simple marking of the same is possible. Moreover, particularly after repair of flaws, it is not necessary to leak-test the entire component, but only the flaw-containing area of the component has to be subjected to another test. Finally, the component to be tested is substantially less influenced by the application of the testing liquid than is the case when immersing the component in a water basin. Besides the application of the testing liquid, it is merely necessary to subject the component to a sufficient temperature increase so that the air contained in the cavities of the component, in particular in the honeycomb core, is expanded to an extent that it will escape via possible leaks and will prompt the testing liquid provided thereabove to form bubbles.

According to a variant of the testing method, at least the component's area to be tested is cooled before being wetted with the testing liquid, and the required temperature increase is achieved merely by the automatic heating of the component to room temperature, optionally with the assistance of sources of heat. Cooling of the component's area to be tested, or of the entire component may be effected by any cooling devices, cooling chambers or cooling tunnels through which the component is moved.

According to a further feature of the invention it is provided that cooling is effected to −30° C. at the most. At such temperatures, a negative influence on the component to be tested is unlikely.

In addition to cooling the component, or as an alternative thereto, at least the component's area to be tested can also be heated after having been wetted with the testing liquid. By this heating, the temperature increase required for the leakage test is achieved.

In doing so, at least the component's area to be tested can be heated by irradiation, in particular infrared irradiation, from the side of the component to be tested that is located opposite the area to be tested. For this purpose, a halogen bulb lamp, e.g., may be positioned at a certain distance of a few cm at the rear side of the component to be tested.

In order to largely prevent a negative influence on the component to be tested, heating preferably is effected to 80° C. at the most.

The variant with cooling of the component prior to wetting of the same with the testing liquid is particularly advantageous for more complex components, in which heating by means of a lamp or other sources of heat from the rear side of the component would not, or would not easily, be possible. Moreover, with the method of cooling the component prior to its wetting with the testing liquid, a simultaneous examination of both surfaces of the component is possible more easily than with the method of heating by means of a lamp or the like.

According to one feature of the invention, the oppositely arranged sides at least of the component's area to be tested are wetted with the testing liquid. By this, a simultaneous checking of both surfaces of the component or of the component's part to be tested is possible.

The locations with bubble formation are marked after having been identified so as to facilitate a subsequent repair of the component. Marking may be by hand, or also automatically. For automatic marking, the area to be tested may, e.g., be scanned with the help of a camera, and bubble formation may be recognized by means of an image processing method, whereupon the mark may be applied to the respective location by a robot arm or the like, e.g.

The testing liquid may, e.g., be applied at least to the component's area to be tested, or also to all the surfaces of the component, by brushing on or by spraying on. Likewise, it is possible for the component to be tested to be guided through a curtain formed by testing liquid flowing down. The respective method of applying the testing liquid must also be-adapted to the shape of the component to be tested or of the component's area to be tested.

After the testing method, the testing liquid is removed by washing, preferably with water. This washing procedure preferably occurs in an automatic wash plant.

Better cleaning results are obtained if the washing procedure occurs under pressure and if the washing procedure possibly is mechanically assisted. This mechanical assistance may e.g. be realized by brushes, sponges or the like.

The testing liquid to be employed during leak-testing components having cavities preferably contains from 10% to 20% dissolved tensides, from 2% to 6% alcohol, and from 75% to 88% water. The percentages indicated are percent by volume. By the indicated composition of the testing liquid, a complete wetting, i.e. a closed film, of the testing liquid is achieved on the component's area to be tested. Moreover, such a testing liquid can be washed off particularly easily and without leaving residues, and does not affect the surface of the component to be tested. Moreover, such a testing liquid is not harmful to the health and to the environment, allowing it to be discharged via the public sewer system. The leak-testing liquid of the type indicated can be applied particularly easily, in particular also by machine, and does not foam during application thereof. The testing liquid is suitable to be used in a temperature range of between −30° C. and 80° C. Moreover, repeating of the testing method after drying of the testing liquid is possible since the testing liquid indicated does not cause a closure of a possible leak.

By the composition, a surface tension of the testing liquid is achieved which allows for the bubbles formed to remain as long as possible above the leak and collapse only little or not at all. This shall be possible particularly also at vertical areas of the components to be tested.

In addition, up to 5% of auxiliaries may be contained in the leak testing liquid. By means of such auxiliaries, various properties can be achieved.

Furthermore, anionic, cationic or non-ionic tensides may be contained.

To increase the stability of the foam-forming testing liquid, preferably trihydric alcohols, in particular glycerol, are contained in the proportions indicated. In this way it is also achieved that the film of testing liquid will remain complete as long as possible on the surface of the component to be tested.

In order to obtain a sufficient contrast on the surface of the component to be tested, coloring pigments may be contained as auxiliaries in the testing liquid. The color will be chosen as a function of the color of the surface of the component to be tested.

Furthermore, also scents may be contained as auxiliaries.

The present invention will be explained in more detail by way of the enclosed drawings.

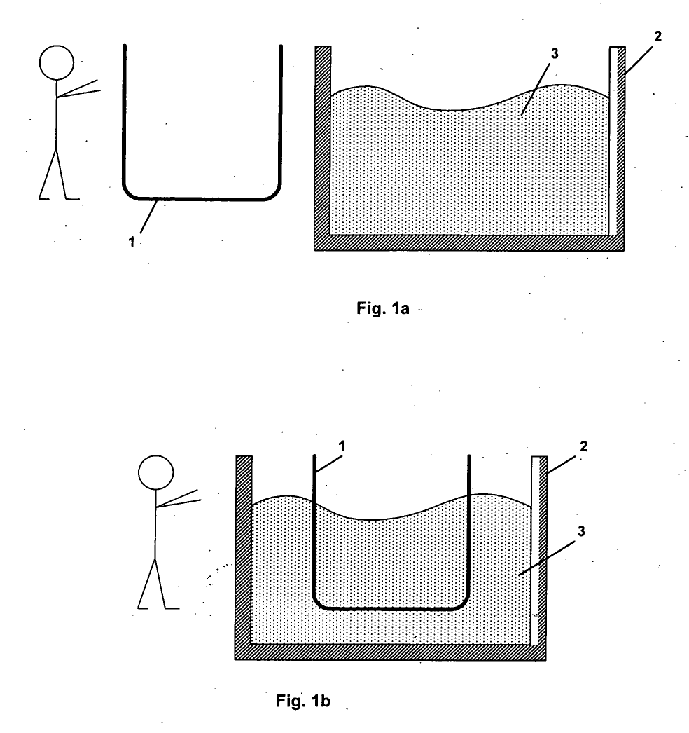

Therein, FIGS. 1a and 1b schematically show the use of a conventional immersion method for leak-testing core composites;

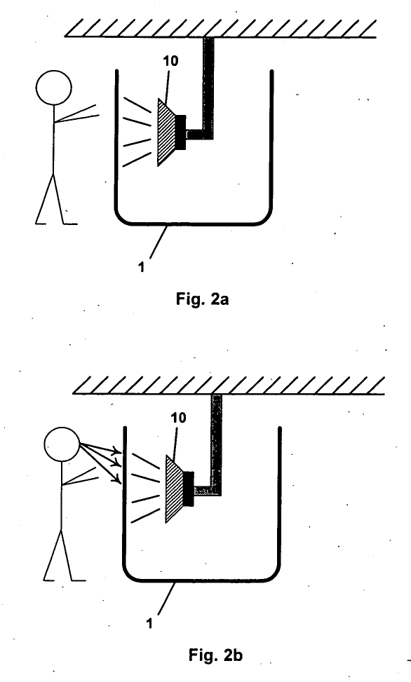

FIGS. 2a and 2b schematically illustrate the method according to the invention for leak-testing components having cavities;

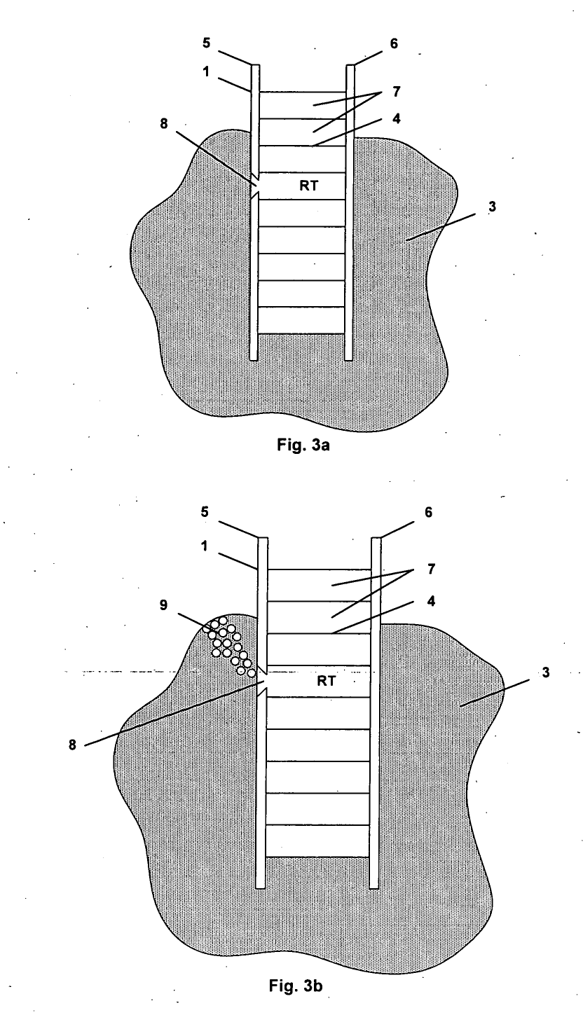

FIGS. 3a and 3b show sectional images of a part of the component to be tested when employing the immersion method according to the prior art;

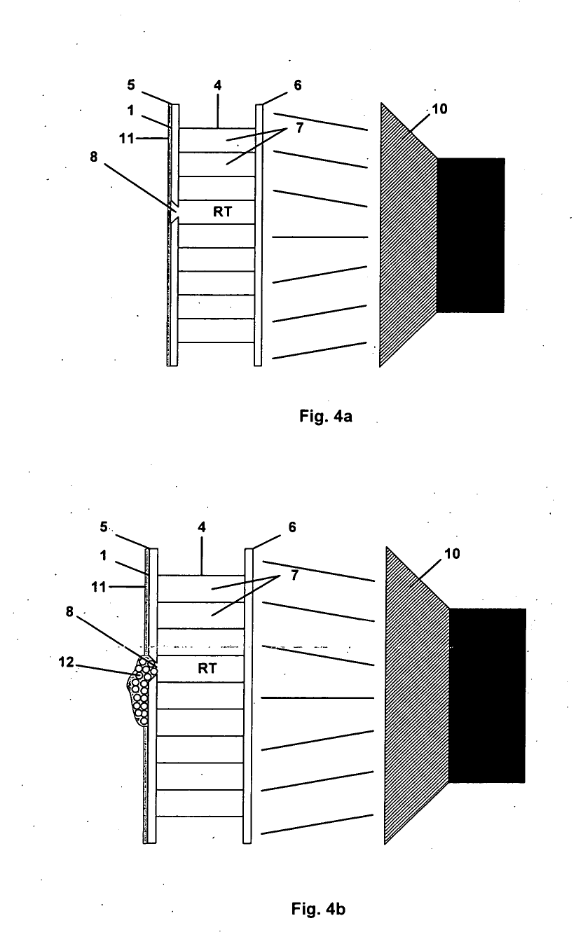

FIGS. 4a and 4b show a portion of the component to be tested in a sectional representation when employing the method according to the invention; and

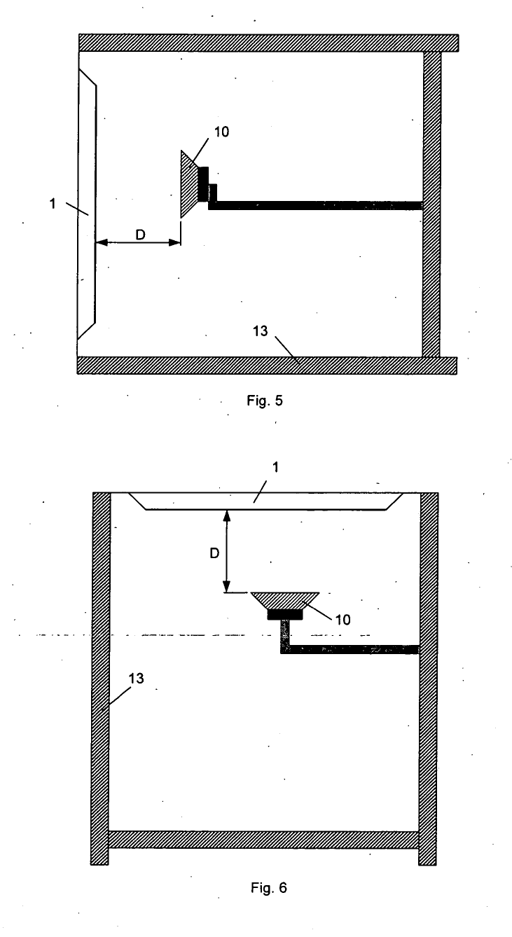

FIG. 5 and FIG. 6 show two possible arrangements of the method according to the invention.

FIGS. 1a and 1b schematically illustrate a method for leak-testing according to the prior art, wherein the component 1 to be tested is introduced into a container 2 with a liquid 3, in particular water. Before its immersion, the component 1 to be tested has room temperature, and subsequently, according to FIG. 1b, it is completely submersed in the liquid 3 which has been heated to 70° C., e.g. In the detailed illustrations according to FIGS. 3a and 3b, a portion of the component to be tested is shown in a sectional representation. The component 1, e.g., consists of a lightweight construction core 4, e.g. a honeycomb core, and cover layers 5, 6, so-called prepreg layers, e.g., of an impregnated carbon fiber fabric, arranged on either side thereof. The lightweight construction core 4 contains hollow chambers 7 which commonly contains air. If the component 1 is now immersed in the liquid 3 which has a higher temperature than the component 1 prior to its immersion in liquid 3, the air present in the hollow chambers 7 will expand. If there exists a leak 8, the expanding air of hollow chamber 7 will escape via this leak 8 and form corresponding air bubbles 9 in the liquid 3. Rising of air bubbles 9 will be observed and identified and marked either under water with an appropriate water-proof marker or the like, or after removal of the component 1 from the basin 2. By the rising air bubbles 9, a precise identification of the leak 8 in most cases will not be possible. Moreover, also when repeatedly leak-testing the component 1, it will always be necessary to completely immerse the entire component 1 in the liquid 3 within the container 2.

FIGS. 2a and 2b schematically show a variant of the method according to the invention, wherein the component 1 is wetted with a testing liquid at least on the area to be tested, whereupon the component 1 is subjected to a temperature increase. This temperature increase from room temperature, e.g. to 80° C., may be effected by means of a lamp 10 which irradiates the component 1 from the rear side of the area to be tested and correspondingly heats the same. From the heating of the area to be tested of the component 1, there will result an expansion of the air contained in the high chambers 7 and possibly an escape via leaks, whereby bubbles will form due to the testing liquid applied, whereby a clear and reliable identification of the faulty sites will become possible.

FIGS. 4a and 4b in sectional representations show a portion of the component 1 to be tested by using the inventive method according to FIGS. 2a and 2b. The surface of the cover layer 5 of the component 1 is completely wetted with a film of the testing liquid 11. By means of a lamp 10, e.g., the component 1 is heated to a temperature which will suffice for an expansion of the air contained in the cavities 7 such that it will escape via leaks 8 possibly present in the component 1. The escaping air forms bubbles 12 from the testing liquid 11. A substantial advantage of the present method consists in that not the entire component 1 needs to be immersed in a liquid, but merely the area to be tested needs to be wetted with a testing liquid 11, and the component 1 has to be subjected to a temperature increase.

FIGS. 5 and 6 show two method arrangements in which the component 1 to be tested is heated by a lamp 10 located at a distance D from the component 1. Lamp 10 may be fastened to a frame 13 and may also be movable so that the entire component 1 can be scanned by lamp 10.

As an alternative to the variants shown in FIGS. 2a, 2b, 4a, 4b and 5 and 6, the component 1 may also be cooled before being wetted with the testing liquid 11, and after having been wetted with the testing liquid 11, it can be warmed merely to room temperature, whereby the air contained in the hollow chambers 7 of component 1 will expand and escape via possible leaks 8. Preferably, such a testing method will be effected automatically in that the components 1 to be tested are guided through corresponding cooling or heating zones, and the testing liquid is automatically applied to the components to be tested. After said testing, the testing liquid 11 will be rinsed off, preferably by means of water, possibly with mechanical assistance. Then the components will be supplied to further processing, they may be provided with an enamel, e.g.

1. A method for leak-testing components having cavities, wherein on at least one side of the component to be tested, at least the area to be tested is completely wetted with a foam-forming testing liquid, characterized in that the component is subjected to a temperature increase and in that subsequently the component's area to be tested is checked for a bubble formation of the testing liquid.

2. A testing method according to claim 1, characterized in that at least the component's area to be tested is cooled before being wetted with the testing liquid.

3. A testing method according to claim 2, characterized in that the cooling is effected to −30° C. at the most.

4. A testing method according to claim 1, characterized in that at least the component's area to be tested is heated after having been wetted with the testing liquid.

5. A testing method according to claim 4, characterized in that at least the component's area to be tested is heated by irradiation, in particular by infrared irradiation, from that side of the component which is located opposite the area to be tested.

6. A testing method according to claim 4, characterized in that the heating is effected to 80° C. at the most.

7. A testing method according to claim 1, characterized in that the oppositely arranged sides at least of the component's area to be tested are wetted with the testing liquid.

8. A testing method according to claim 1, characterized in that the sites exhibiting bubble formation are marked.

9. A testing method according to claim 1, characterized in that the testing liquid is applied by brushing to at least the component's area to be tested.

10. A testing method according to claim 1, characterized in that the testing liquid is applied by spraying to at least the component's area to be tested.

11. A testing method according to claim 1, characterized in that after said testing, the testing liquid is removed by washing, preferably with water.

12. A testing method according to claim 11, characterized in that the washing process is effected under pressure.

13. A testing method according to claim 11, characterized in that the washing process is mechanically assisted.
